(19) United States
(12) Patent Application Publication
(10) Pub. No.: US 2018/0298430 A1
(43) Pub. Date: Oct. 18, 2018

(54) GENOMIC DNA MUTATION ASSAYS AND USES THEREOF

(71) Applicant: The Board of Regents, The University of Texas System, Austin, TX (US)

(72) Inventor: Hsi-Ming Wang, Houston, TX (US)

(21) Appl. No.: 15/965,856

(22) Filed: Apr. 28, 2018

Related U.S. Application Data
(60) Provisional application No. 62/464,966, filed on Feb. 28, 2017.

(51) Int. Cl. C12Q 1/6827; C12Q 1/6869

(52) U.S. Cl. CPC C12Q 1/6827; C12Q 1/6869

(57) ABSTRACT

Described herein are techniques and methods to analyze mutations in genomic DNA. The techniques and methods can be used to diagnose disease in a subject.

### CROSS-REFERENCE TO RELATED APPLICATIONS

This application claims the benefit of and priority to co-pending U.S. Provisional Patent Application No. 62/464,966, filed on Feb. 28, 2017 entitled “muSeq: A high-throughput method for identifying mutations,” the contents of which is incorporated by reference herein in its entirety.

### BACKGROUND

Mutations can be the primary cause of genetic disorder and cancer. The ability to detect mutations in the genome can hold the key to diagnosis, treatment, and/or prevention of the disease. As such, there exists a need for improved techniques to identify genomic mutations.

### SUMMARY

Described herein are aspects of a method of identifying genomic DNA mutations, the method that can include the steps of denaturing double stranded (ds) genomic DNA fragments to form single stranded (ss) genomic DNA fragments; annealing the ssDNA to form homoduplex DNA fragments and heteroduplex DNA fragments; enzymatically identifying a heteroduplex DNA fragment having a base pair mismatch by binding the heteroduplex DNA fragment having a base pair mismatch; separating the heteroduplex DNA fragment having a base pair mismatch from the homoduplex DNA fragments and any heteroduplex DNA not having a base pair mismatch to obtain an isolated heteroduplex DNA fragment having a base pair mismatch; ligating a sequencing adaptor to an end of each strand of the isolated heteroduplex DNA fragment having a base pair mismatch; digesting the adaptor-ligated isolated heteroduplex DNA fragment having a base pair mismatch with an exonuclease capable of degrading ds DNA removing the enzyme from the adaptor-ligated heteroduplex DNA fragment having a base pair mismatch and forming an adaptor-ligated ss DNA; circligating the adaptor-ligated ss DNA; PCR amplifying the circle-ligated adaptor-ligated ssDNA to form a sequencing library; and sequencing the sequencing library using a next generation sequencing method. The step of enzymatically identifying a heteroduplex DNA fragment having a base pair mismatch can include binding the heteroduplex DNA fragment having a base pair mismatch with an enzyme that specifically binds the heteroduplex DNA fragment at mismatched base pair. The enzyme can be mutS. The mutS can include an affinity purification tag. the affinity purification tag can be selected from the group of: a his-tag, a chitin binding protein tag, maltose biding protein tag, a strep-avidin tag, a glutathione-S-transferase tag, FLAG-tag, V5-tag, VSV tag Myc-tag, HA0tag, Spot tag, NE tag and any combination thereof. The step of separating can be performed by affinity precipitating out the heteroduplex DNA with an antibody or affinity purification complex that specifically binds the mutS or affinity purification tag. The exonuclease can be T5 exonuclease. The next generation sequencing method can be a sequencing by ligation method or a sequencing by synthesis method. The sequencing by synthesis method can be pyrosequencing. ds genomic fragments can be from genomic DNA of a single subject. The method can further include the step of digesting the genomic DNA with a restriction enzyme to generate the ds genomic fragments. The step of sequencing can form sequencing reads. The method can further include the step of mapping the sequencing reads to a genome build. the method can further include the step of identifying mismatch variants and estimating relative allele frequency (RAF) and identifying somatic mutations from parental mutations. The genomic DNA mutation can be a somatic mutation.

Also described herein are aspects of a method of identifying genomic DNA mutations that can include the steps of denaturing double stranded (ds) genomic DNA fragments to form single stranded (ss) genomic DNA fragments; annealing the ssDNA to form homoduplex DNA fragments and heteroduplex DNA fragments; identifying a heteroduplex DNA fragment having a base pair mismatch to form an identified heteroduplex DNA fragment; separating the heteroduplex DNA fragment from the unprotected homoduplex DNA fragments; ligating an adaptor to an end of each DNA strand in the heteroduplex DNA fragment to form an adaptor-ligated DNA; PCR amplifying the adaptor-ligated DNA to form a sequencing library; and sequencing the sequencing library using a next generation sequencing method. The step of sequencing can form sequencing reads and further comprises mapping the sequencing reads to a genome build. The method can further include the step of identifying mismatch variants and estimating relative allele frequency (RAF) and identifying somatic mutations from parental mutations. The next generation sequencing method can be a sequencing by ligation method or a sequencing by synthesis method.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 is a flow chart that can generally describe the genomic DNA mutation assay described herein.

FIG. 2 is a flow chart demonstrating aspects of the genomic DNA mutation assay described herein. Color code of each box indicates the experimental condition of each step (black: in solution reactions, dark grey: DNA bound to solid beads, light grey: computational steps). The number labeled on the top left corner of each box and the arrows linking between boxes indicate the order and the flow of individual steps of the invention. Some schematics are provided in the subsequent figures to help visualizing the invention. These figures are meant to be illustrative and not to be considered as restrictions of the invention or as exact depictions of the methods described herein.

FIG. 3 is a schematic depiction of the formation of heteroduplex genomic DNA fragments. Genomic DNA fragments, prepared from restriction enzyme digestion, are denatured and re-annealed through temperature gradients. Color code indicates the parental origin of genomic DNA fragments and the presence of somatic mutations on the fragment (dark grey and light grey indicates parental origins, black indicates fragments containing somatic mutations).

FIG. 4 is a schematic depiction of an embodiment of enzyme identification of heteroduplex DNA fragment having a base pair mismatch, protection of these heteroduplex DNA fragments, and affinity purification and separation of protected from unprotected duplex DNA fragments.

FIG. 5 is a schematic depiction of adaptor ligation to heteroduplex DNA fragments and the subsequent exonulease treatment. These treatments are for preparing the fragments for high-throughput sequencing and for removing residual homoduplexes (homoduplexes are not shown in the figure).

FIG. 6 is a schematic depiction of sequencing library construction following the mutS protected exonuclease digestion step. These DNA fragments contain either germline variants or somatic mutations. In some aspects, after the enzyme and/or affinity purification complexes are removed, the single stranded DNA fragments can be prepared for sequencing, such as by being circularized.

FIG. 7 is a diagram illustrating the features of a sequencing adaptor. As a non-limiting example, FIG. 7 shows an adaptor designed for the Illumina TruSeq sequencing platform.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, genetics (including molecular genetics), organic chemistry, biochemistry, physiology, cell biology, cancer biology, and the like, which are within the skill of the art. Such techniques are explained fully in the literature.

Definitions

As used herein, As used herein, “about,” “approximately,” and the like, when used in connection with a numerical variable, can generally refers to the value of the variable and to all values of the variable that are within the experimental error (e.g., within the 95% confidence interval for the mean) or within +/−10% of the indicated value, whichever is greater.

As used herein, “antibody” can refer to a glycoprotein containing at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hyper variability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen.

As used herein “cancer” can refer to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basal cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, hary cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodyspastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sézary syndrome, skin cancer, small intestine cancer, large intestine cancer (colon cancer), soft tissue sarcoma, T-cell lymphoma, throat cancer, oropharyngeal cancer, nasopharyngeal cancer, hypoharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, B cell lymphoma, post-transplant lymphoproliferative disorder, and Wilms Tumor.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “DNA molecule” can include nucleic acids/polynucleotides that are made of DNA.

As used herein, “homoduplex DNA” can refer to double stranded DNA where both strands are from the same source.

As used herein, “heteroduplex DNA” can refer to double stranded DNA where each strand is from a different source. Heteroduplex DNA can include one or more base-pair mismatches between each strand.

As used herein, “nucleic acid,” “nucleotide sequence,” and “polynucleotide” can be used interchangeably herein and can generally refer to a string of at least two base-sugar-phosphate combinations and refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same term can be applied to the arrangement of coding sequences and/or transcription control elements (e.g. promoters, enhancers, and termination elements), and/or selectable markers in an expression vector. “Operatively linked” can also refer to an indirect attachment (i.e. not a direct fusion) of two or more polynucleotide sequences or polypeptides to each other via a linking molecule (also referred to herein as a linker.

As used herein, “purified” or “purify” can be used in reference to a nucleic acid sequence, peptide, polypeptide, or other complex or molecule that has increased purity relative to the natural environment or other environment.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “separated” can refer to the state of being physically divided from the original source or population such that the separated compound, agent, particle, or molecule can no longer be considered part of the original source or population.

As used herein, the term “specific binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁶ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, “subject,” “individual,” or “patient” can refer to a vertebrate organism, such as a mammal (e.g. human, companion animal, domestic livestock, etc.). “Subject” can also refer to a cell, a population of cells, a tissue, an organ, or an organism, preferably to human and constituents thereof.

Discussion

Mutations are the primary cause of genetic disorder and cancer. The ability to detect mutations in the genome holds the key to diagnosis, treatment, and prevention of the disease.

Recent developments in novel sequencing technology have made efforts in sequencing hundreds of thousands of human genomes a reality. However, it remains challenging to identify rare somatic mutations. Somatic mutations often present only in a trace amount in the sample. Efforts in using high-throughput sequencing to identify somatic mutations often waste majority of the sequencing reads on regions without mutations. Moreover, the low occurrence rate of somatic mutations makes it technically challenging to distinguish true mutations from sequencing errors.

An outstanding challenge concerns the detection of circulating tumor DNA (more precisely, detection of somatic mutations present in circulating tumor DNA). Reliable detection of circulating tumor DNA could provide early diagnosis for cancer patients. Because circulating tumor DNA molecules are only present in a trace amount in body fluids, to avoid wasting most of the sequencing effort to normal DNA and to increase statistical power to distinguish true mutations from sequencing errors, available methods for detecting circulating tumor DNA focus on either reducing the search space (e.g. sequencing only a predefined set of regions) or enriching for the presence of circulating tumor DNA in test samples (e.g. isolating circulating tumor cells). While reducing search space by focusing only on known somatic mutations (which is often patient specific) presents an attractive (less technical) direction, it requires prior knowledge on the presence of a certain set of mutations on the patient. This requirement not only renders the aforementioned approach patient specific (therefore harder to scale), but also renders it not applicable for prevention and diagnosis as no prior knowledge would be available on mutations present in potential patients.

With the aforementioned deficiencies in mind, described herein are methods that can include forming heteroduplex genomic DNA fragments, identifying base pair mismatches in the heteroduplex genomic DNA fragments, separating the heteroduplex genomic DNA fragments with a base pair mismatch from homoduplex genomic DNA and heteroduplex genomic DNA without a base pair mismatch, generating a sequencing library from the separated heteroduplex genomic DNA fragments with a base pair mismatch, and using a next generation sequencing method to sequence the library. The sequence reads generated from sequencing the library can be mapped to a reference genome, mismatch variants can be identified, and the relatie allele frequency (RAF) can be estimated and used to distinguish somatic mutation from parental polymorphisms. The somatic mutation information can be used to determine disease or other physiologic state. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Identification of Mutations in Genomic DNA

The methods described herein can direct the power of next generation sequencing to the regions of the genome containing somatic mutations and/or germ line polymorphisms. Current mutation analysis methods based such as a traditional DNA mismatch assay require two separate strands: a “reference” strand and a “sample” strand from two different sources. Thus, traditional DNA mismatch analysis are unsuitable for identification of unknown mutations in an individual because they require prior knowledge of the presence of a certain set of mutations in the subject. The methods described herein are such that the “reference” strand and a “sample” strand can be from the same source, thus making it practical for the identification of unknown mutations in an individual subject.

The methods described herein further include the use of massively parallel sequencing methods (also referred to as next generation sequencing methods herein) to determine the frequency of DNA mutations, such as somatic mutations, identified in a subject. By analyzing the frequency of a mutation or a set of mutations across an individual's genome, a disease or other physiological state of the subject can be determined.

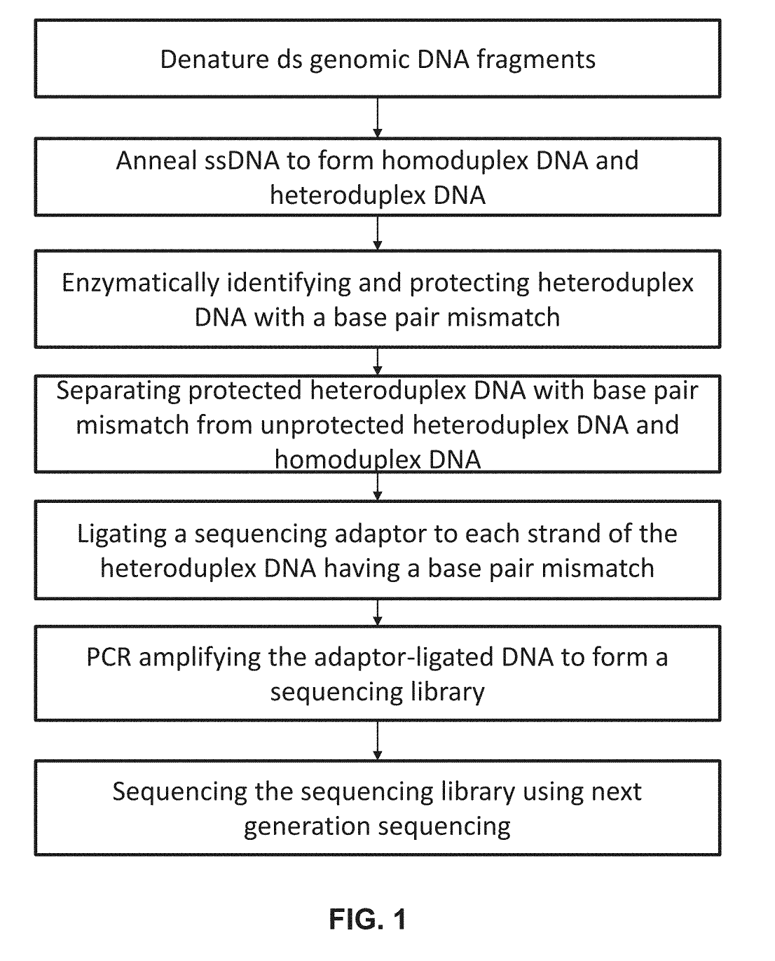

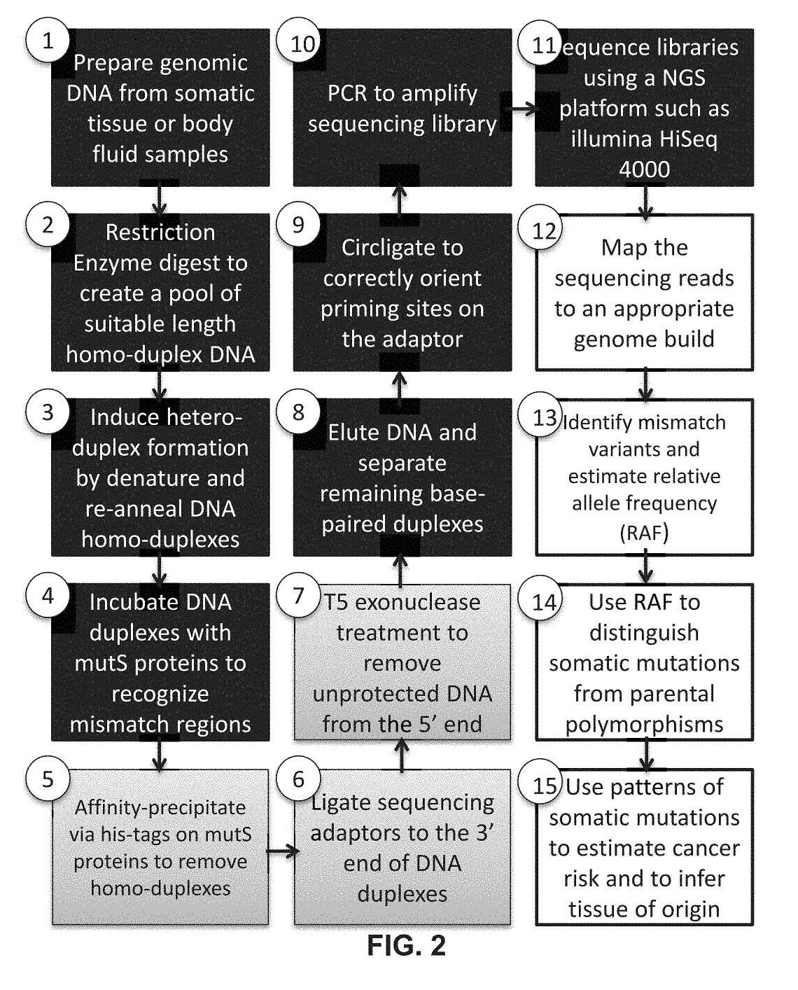

As shown in FIGS. 1-2, the method can include denaturing double stranded (ds) genomic DNA fragments to form single stranded (ss) genomic DNA fragments. Denaturing can be performed by heating the ds genomic DNA fragments at a temperature beyond the melting point of the DNA fragment. In some aspects, the ds genomic DNA fragments can be heated to a temperature of about 90 to about 95° C. for about 1 to about 20 minutes to denature the ds genomic DNA fragments.

The ds genomic DNA fragments can be obtained by digesting whole genomic DNA with restriction enzyme(s) that are capable of yielding DNA fragments. In some aspects, the restriction enzyme can produce fragment ends suitable for sequencing adaptor ligation. The restriction enzyme chosen can be one that produces a suitable average fragment as well as the desired fragment end (e.g. 3′ or 5′ overhang with appropriate base pairs to allow for sequencing adaptor ligation). In some aspects the restriction enzyme can produce 5′ AT overhangs on the resulting ds genomic DNA fragments. In some aspects, the ds genomic DNA fragments can be obtained by physical fragmentation. In some aspects, the physical fragmentation approach chosen is sonication, such as using a Covaris M220 Focused-ultrasonicator. In some aspects, adenylation or tailing of the resulting ds genomic DNA fragments can be completed. The genomic DNA fragments can range in size from 25 bp to 1000 bp. In some aspects, the average genomic ds DNA fragment size after restriction enzyme digest is about 200 bp. In some aspects, the restriction enzyme can be MseI. In some aspects, the restriction enzyme can be either NdeI, AseI, BfaI, or CViQI. In some aspects, a mixture of the above motioned restriction enzymes could be used. In some aspects, alternative restriction enzymes could be used provided with corresponding changes in the design of the adaptor for ligation. The ds genomic DNA can be obtained from any cell(s), tissue, and/or bodily fluid from a subject using any DNA extraction and preparation method for that particular cell, tissue, or bodily fluid. Such methods are generally known in the art and reagents are commercially available. In some aspects, the ds genomic DNA can be prepared by using phenol-chloroform extraction followed by ethanol precipitation. In some aspects, the ds genomic DNA can be prepared from tissue samples using either DNAzol reagent or PureLink Genomic DNA Mini Kit. In some aspects, the ds genomic DNA can be prepared from blood samples using QIAamp DNA Blood Midi Kit.

After the ds genomic DNA fragments have been denatured into ss genomic DNA fragments, the ss genomic DNA fragments can be annealed to form homoduplex DNA fragments and heteroduplex DNA fragments. Annealing can be performed by cooling the denatured DNA through a temperature gradient. While not being bound to theory, the ss genomic DNA molecules containing germline variants have a statistical probability of 50% of forming homoduplexes upon annealing and ss genomic DNA fragments having somatic mutations are very unlikely to form homoduplexes upon annealing. The probability of homoduplex formation during annealing is mostly determined by the allele frequency of the mutation in the ss genomic DNA fragment pool.

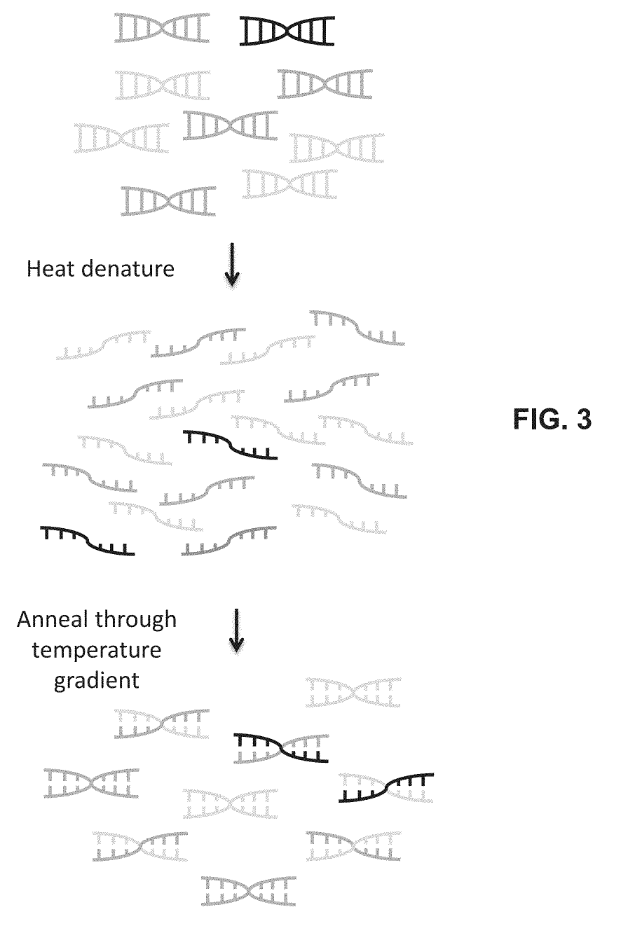

As shown in FIG. 3, in most scenarios, especially for samples derived from an inbred individual, the majority of DNA duplex fragments will contain neither germline variants nor somatic mutations. Those DNA duplexes, which contain no genetic variants, are not depicted in this diagram. Homoduplex fragments containing either germline variants (dark grey or light grey) or somatic mutations (black) are heat denatured to become two single-stranded DNA molecules and then re-annealed to form duplex DNA fragments. While single-stranded DNA molecules containing germline variants have a 50% chance of forming homoduplexes (dark grey-dark grey or light grey-light grey) upon re-annealing, single-stranded DNA molecules containing somatic mutations (dark grey) are very unlikely to form homoduplexes upon re-annealing. The probability of homoduplex formation upon re-annealing is largely determined by the allele frequency of the mutation in the pool.

After annealing, heteroduplex DNA fragments having a base pair mismatch can be identified. In some aspects, heteroduplex DNA fragments having a base pair mismatch enzymatically. In some aspects, enzymatic identification can be conducted using an enzyme that is capable of recognizing (e.g. binding or cleaving to mismatched DNA) can be contacted with the heteroduplex and homoduplex DNA fragments. Where there is a base pair mismatch in the heteroduplex DNA, the enzyme can bind, cleave and/or otherwise act on the DNA thus identifying the base pair mismatch.

In some aspects, the enzyme can be mutS, Surveyor (e.g. CEL2), or T7E1. In some aspects, the enzyme can be T4E7, CEL1, or ENDO1. In some aspects, the enzyme can remain bound to the heteroduplex DNA at the point of base pair mismatch. In some aspects, such as with mutS, the position on the heteroduplex DNA fragment having the base pair mismatch as wells as some of the base pairs up and downstream from the base pair mismatch are covered by the bound enzyme. Thus in this way these base pairs of the fragment can be protected in subsequent steps of the method, such as DNA digestion. Thus, in some aspects the step of enzymatically identifying the heteroduplex DNA fragment having a base pair mismatch can result in protected heteroduplex DNA fragment(s) and, thus implicitly, unprotected homoduplex DNA fragment(s).

The protected heteroduplex DNA fragment(s) can be separated from the unprotected homoduplex DNA fragments and unprotected heteroduplex fragments (e.g. those not having base pair mismatches). In some aspects the step of separating the protected heteroduplex DNA fragment(s) can be separated from the unprotected homoduplex DNA fragments and unprotected heteroduplex fragments can be performed using an affinity purification method.

In some aspects the affinity purification method can be immunoprecipitation. In some aspects, an antibody that can specifically bind the enzyme bound to the heteroduplex DNA fragment(s) can be used to identify those duplex DNA fragments that are bound to enzyme. The antibody bound the protected heteroduplex DNA fragment(s) can be removed from the other DNA duplexes using an immunoprecipitation technique. By way of a non-limiting example, if the antibody is an IgG antibody, the pool can be exposed to an IgG affinity column, which will bind the IgG antibody and thus the protected heteroduplex DNA fragment(s) while allowing the rest of the duplex DNA to flow through. The column bound antibody bound the protected heteroduplex DNA fragment(s) can then be recovered from the column and be substantially free of the other duplex DNA fragments. The antibody can be one that binds to the enzyme used. In other aspects, the enzyme can be modified to include a purification tag that can be recognized by an antibody. Thus in those cases, the antibody can recognize the modified enzyme via binding to the tag. Suitable tags include, but are not limited to, a his-tag, a chitin binding protein tag, maltose binding protein tag, a strep-avidin tag, a glutathione-S-transferase tag, FLAG-tag, V5-tag, VSV tag Myc-tag, HAOtag, Spot tag, NE tag and any combination thereof.

In some aspects, an enzyme that is capable of binding ds DNA at a base pair mismatch can be modified to contain an affinity purification tag. Suitable tags include, but are not limited to, a his-tag, a chitin binding protein tag, maltose binding protein tag, a strep-avidin tag, a glutathione-S-transferase tag, FLAG-tag, V5-tag, VSV tag Myc-tag, HAOtag, Spot tag, NE tag and any combination thereof. Instead of an antibody binding the enzyme or affinity purification tag as previously described, non-antibody reagents that bind the tag can be used to bind to and subsequently purify the protected heteroduplex genomic DNA complexes. By way of a non-limiting example, the enzyme can be modified to contain a His-tag (e.g. a His or poly-His tag). A complex containing nickel conjugated to a magnetic bead can be used to bind the His-tag. The pool of DNA complexes can then be exposed to a magnetic field to separate out the protected and unprotected genomic DNA duplexes. The protected heteroduplexes will be bound by the nickel-magnetic bead and thus will be separated from the unprotected duplex genomic DNA fragments that are not as they will separate from one another in the magnetic field.

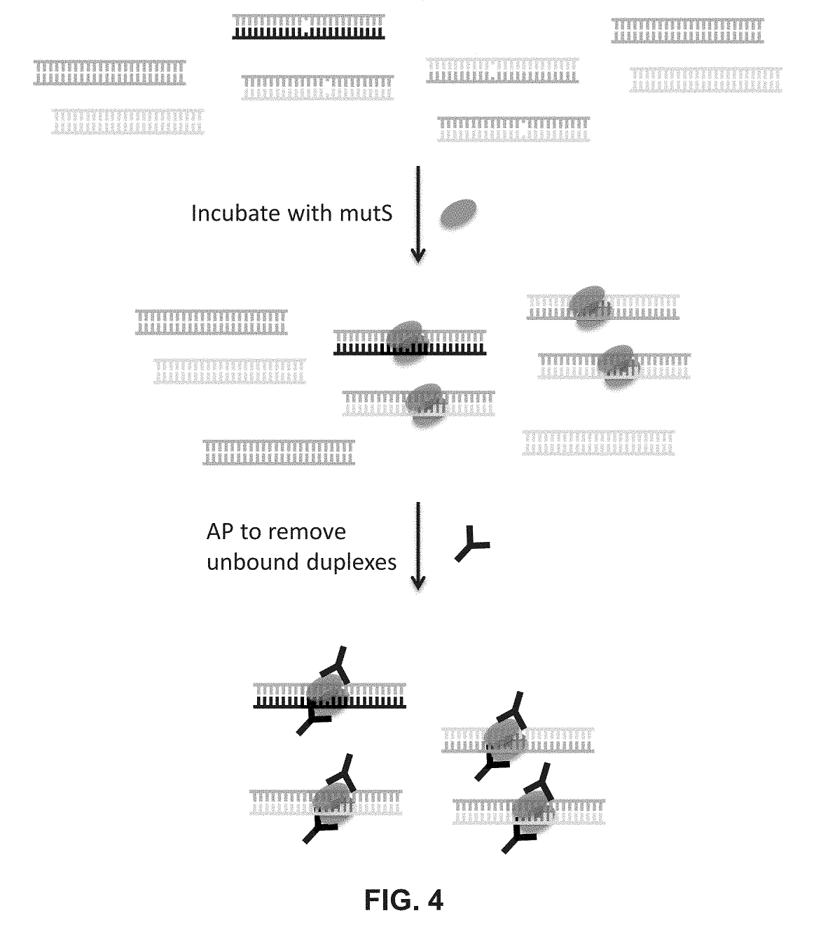

As shown in FIG. 4, in some aspects of mutS recognizes mismatches on double stranded DNA and can be used to affinity purify of mutS-heteroduplex complexes from homoduplexes. The elliptical shaped objects represent affinity purification tagged (e.g. histidine-tagged) modified mutS proteins. Two mutS proteins combined to form a protein complex, which binds specifically to mismatches on double stranded DNA molecules, is used to distinguish heteroduplex DNA fragments from homoduplexes. These mismatches could arise from either hybridization between fragments containing different germline variants (light grey-dark grey) or hybridization between fragments that contain somatic mutations and fragments without somatic mutations (black-dark grey or black-light grey). The Y shaped objects represent nickel-conjugated magnetic bead (which can also be antibodies in other aspects). These beads can be used to pull down histidine-tagged mutS proteins and heteroduplex DNA fragments that are bound by the mutS protein complexes.

In other aspects, the enzyme that can recognize DNA base pair mismatch can cleave the DNA at or near the site of mismatch (e.g. Surveyor enzyme). In these aspects, the cleaved heteroduplex DNA is not necessarily protected by the binding of the enzyme to the ds DNA at the site of bp mismatch. Instead, oligonucleotides that can include a label on one or more nucleotides can be ligated to the cleaved heteroduplex DNA. The label can facilitate identification and affinity purification of the desired heterodupelex DNA. For example, the label can be biotin, which can facilitate purification over a streptavidin column similar to the affinity purification methods described above. This obviates the need for prior knowledge of the flanking nucleotide sequence or the sequence of the variant gene mutation.

In other aspects, non-enzymatic based methods can be used to identify the heteroduplex DNA fragments containing base pair mismatches. Base pair mismatches can be cleaved chemically, using for example, NH₂OH/KMnO₄ to label mismatched C/T in combination with piperidine to break the phosphodiester bond at the chemically labeled sites. In some aspects, heteroduplex mismatch regions such labeled chemically may be isolated using solvent extractions, based on the alterations that such chemicals induce into the nucleotide bases. Labeled oligonucleotides can be used for identification and purification of the resulting heteroduplex fragments as previously described.

After separation, if desired, the affinity reagents (e.g. the antibody or nickel-magnetic bead) can be removed from the protected heteroduplex genomic DNA fragments or labeled genomic DNA fragments.

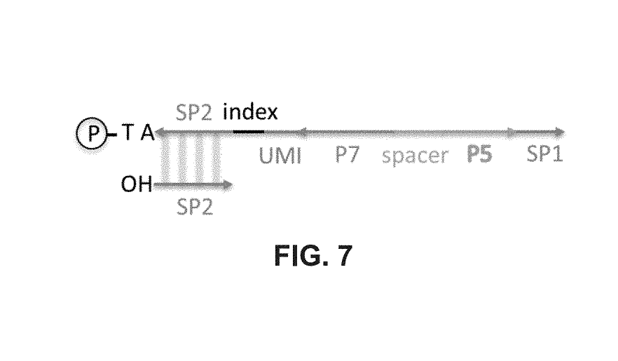

After separation, a sequencing adaptor can be ligated to an end of each DNA strand in the protected heteroduplex DNA fragment or the labeled heteroduplex DNA fragment to form an adaptor-ligated heteroduplex DNA. The adaptor can be configured to bind the 5′ end and/or the 3′ end. The adaptor can be configured to be suitable for a next generation sequencing method. In some aspects, the adaptor can be suitable for Illumina sequencing and can be adapted for hybridization to the illumine flow cell and bridge amplification. The adaptor can include one or more sequencing primer binding sequences. The adaptor can include one or more PCR amplification primer binding sites. These can be used to PCR amplify the heteroduplex DNA fragments for generation of a sequencing library. The adaptor can include a unique molecular identifier (UMI) sequence. The UMI(s) can be included to allow for identification of individual template molecules in the sequencing library. The adaptor can include one or more index sequences. The index sequence(s) can be included to allow for multiplex sequencing. The adaptor can include one or more spacer sequences that can be between the one or more sequencing primer binding sequence(s) and/or UMI sequence(s). The UMI is a short stretch of fully degenerate sequence that provides unique identifiers for individual template molecules. The adaptor can be double stranded in a portion. For example the adaptor can have a 3′ overhang and be ds at the 5′ end as shown in FIG. 7. The inclusion of a ds end can increase the efficiency of adaptor ligation to the protected heteroduplex DNA fragments.

FIG. 7 shows an adaptor designed for use with the Illumina TruSeq sequencing platform. In other aspects, where a different sequencing platform is used, the adaptor can be designed for that specific platform. The adaptor can be designed and configured to include any feature necessary for downstream PCR amplification and sequencing and can be designed on one strand of the adaptor (the adaptor strand). In some aspects, the opposite strand can only have a short stretch of sequence that is reverse complementary to the 5′ end of the adaptor strand. This double-stranded segment is designed for efficient ligation to the heteroduplex DNA fragments. In some aspects an AT overhang at the double-stranded end of the adaptor can be included and can be designed specifically for ligation to Mse I digested DNA fragments. The overhang can be changed to accommodate a different restriction enzyme that is used during the step of fragmenting the genomic DNA.

The 5′ end of the adaptor strand can ligate to the 3′ end of either strand or both strands of a heteroduplex DNA fragment. The PCR priming sites (e.g. P5 and P7 sites) can be oriented away from each other. The orientation of PCR priming sites in this way are configured for an inverse PCR reaction with PCR (e.g. P5 and P7) primers to amplify circular templates, which can result in creating linear sequencing libraries. These libraries can then contain the target inserts and all of the features on the adaptor, except the spacer. Similarly, the sequencing priming sites (e.g. SP1 and SP2) can be oriented away from each other on the adaptor. In addition, SP1 and SP2 priming sites are placed at the end of the adaptor. After circularization, this configuration can put one of the sequencing priming sites (e.g. SP1) close to the site of the mismatch. An index sequence (about timers of specific sequences) can be included on each adaptor for multiplex sequencing. A unique molecular identifier (UMI) can also be included to identify PCR products that are amplified from the same template molecule.

It will be appreciated that the specific configuration and sequence of the adaptor will be dependent on the next generation sequence platform being used to sequence the library that is formed in subsequent steps. Suitable methods and commercially available services for adaptor design will be appreciated by those of ordinary skill in the art in view of this description. Once designed the adaptors can be made using de novo DNA synthesis techniques.

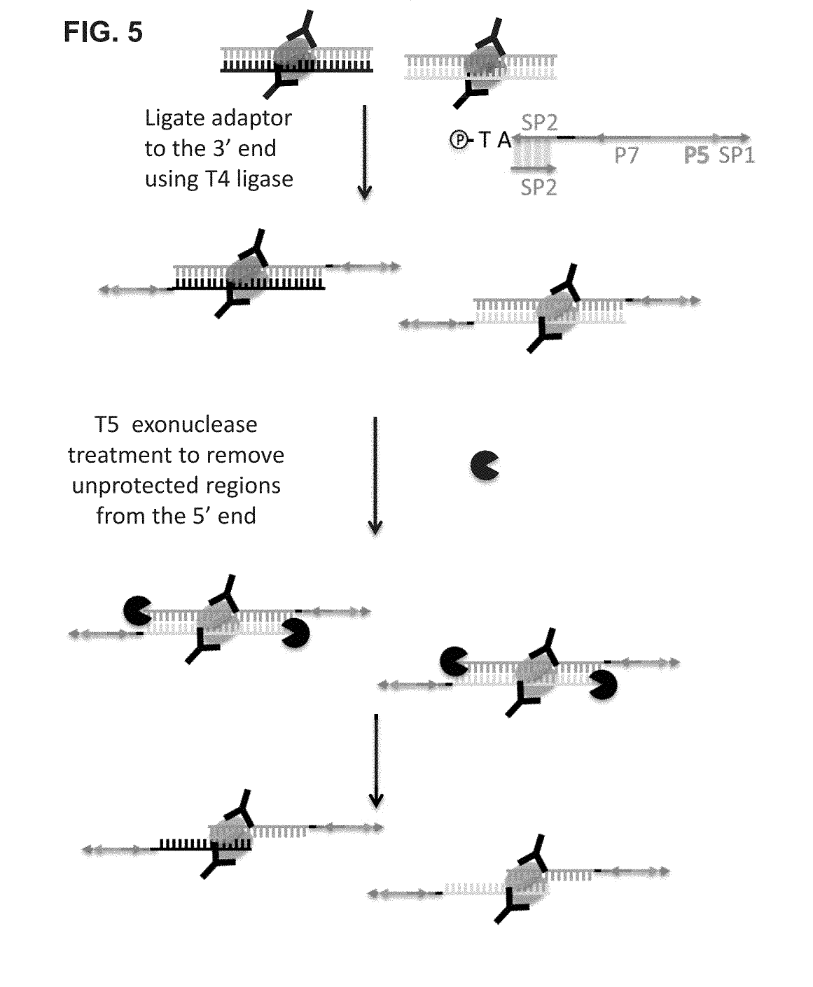

After adaptor ligation, the heteroduplex DNA can be prepared for sequencing. In some aspects, exonuclease digestion can be performed to remove the unprotected ds DNA (e.g. the base pairs that are not covered by the bound enzyme) on the protected heteroduplex DNA fragment. In some aspects, an exonuclease having exonuclease activity in the 5′ to 3′ direction can be used to degrade the unprotected base pairs as shown in FIG. 5. Suitable exonucleases include, but are not limited to, T5 exonuclease, lambda exonuclease, or T7 exonuclease. In some aspects, an exonuclease having exonuclease activity in the 3′ to 5′ direction can be used to degrade the unprotected base pairs provided that corresponding adjustments were made to the adaptor design. Suitable exonucleases that possess exonuclease activity in the 3′ to 5′ direction include, but are not limited to, exonuclease III and T4 DNA polymerase.

As shown in FIG. 5, the pacman shaped objects represent a suitable exonuclease (e.g. T5 exonuclease). The exonucleases degrade double stranded DNA substrates in the, for example based on T5 exonuclease, 5′ to 3′ direction. For a mutS-heteroduplex (or other suitable enzyme) complex, this degradation will stop at the edge of the mutS protein (or other suitable enzyme) complex. In the case of T5 exonucleases, this property of T5 exonucleases preserves for each DNA fragment the region 3′ to the protein complex and therefore increases the mappability of the sequencing reads derived. This same property of T5 exonucleases also leaves single-stranded ends, which are poor substrates for DNA ligase. Ligation of sequencing adaptors is therefore executed before T5 exonuclease or other exonuclease digestion. Details on adaptor design are in FIG. 7 and elsewhere herein. The Y shaped objects bound to enzyme-heteroduplex complexes seen throughout this figure indicate that these steps are executed on duplex DNA fragments that are bound to affinity purification complex or molecule.

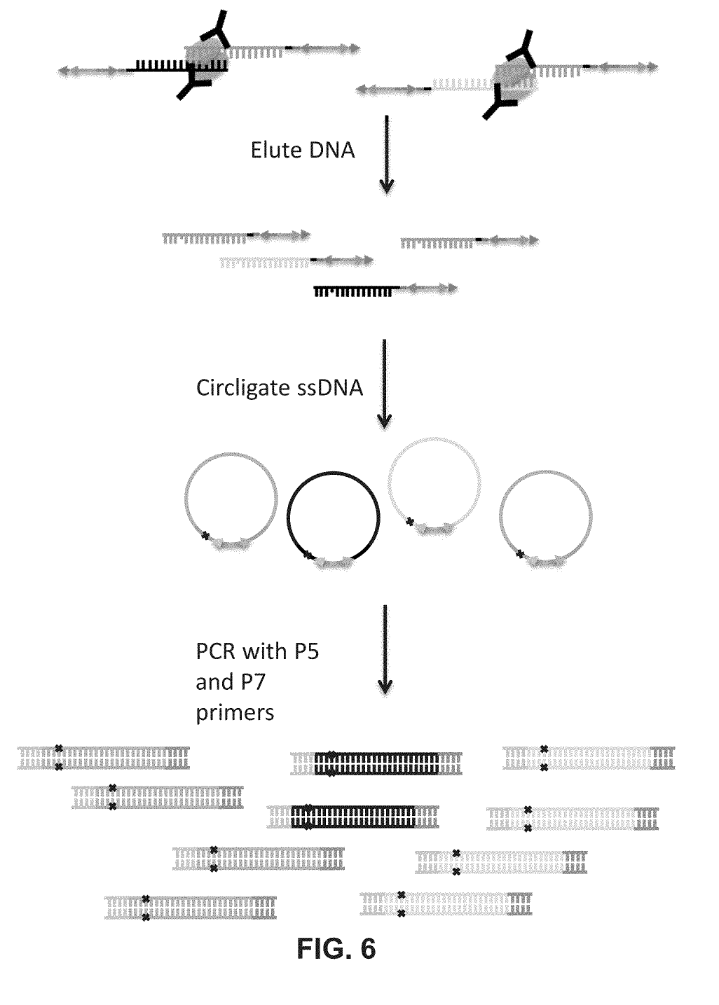

After adaptor ligation and digestion, the adaptor ligated protected heteroduplex DNA can be amplified using a suitable polymerase chain reaction (PCR) technique to form a sequencing library. In some aspects, after adaptor ligation the adaptor-ligated protected heteroduplex DNA fragments can be circligated. A circligase enzyme can be used to circularize the adaptor-ligated protected heteroduplex DNA fragments. Circularization can orient the PCR and/or sequencing primers appropriately as shown in FIG. 6. As shown in FIG. 6, for example, circularization orients the PCR primer sites (e.g. P5 and P7 priming sites) in such directions that PCR amplification would go outwards and result in amplification of the DNA fragments captured by the enzyme (e.g.) mutS into linear sequencing libraries. The black crosses, either on circular templates or on linear PCR products, indicate the sites of mismatches recognized by the enzyme used to identify the base pair mismatch (e.g. mutS enzyme).

The sequencing library can be sequenced using a next generation sequencing method to produce sequencing reads. In some aspects, the next generation sequencing method can be a sequencing by ligation method or a sequencing by synthesis method. In some aspects the sequencing by synthesis method can be pyrosequencing. Suitable next generation sequencing platforms can include, but are not limited to, 454 sequencing, SOLiD sequencing, Illumina sequencing, Ion Torrent sequencing, Ion proton sequencing, and MinION sequencing.

The sequencing reads can be mapped to an appropriate genome build using a suitable alignment programs generally known in the art. After alignment of the sequencing reads to the genome, the occurrence of each mutation in the sample can be estimated by counting the number of sequencing reads that carry the exact mutation. This can then be converted to a relative allele frequency by comparing the number of sequencing reads carrying each somatic mutation to the number of sequencing reads carrying germline variants. The numbers can be normalized or standardized as needed to account for e.g. amplification efficiencies, enzyme binding bias for different mismatch types, uneven sequencing coverage. The normalization and/or standardization can be based on the shared allele frequency among germline variants (50%) and the potential sharing of sequencing coverage between proximal genomic regions.

The allele frequencies of somatic mutations, the genome wide distributions of somatic mutations, and/or other patterns of somatic mutations identified by the sequencing method described above can be applied to determine disease, disease state, or other physiological condition or state. For example, the somatic mutations, the genome wide distributions of somatic mutations, and/or other patterns of somatic mutations identified by the sequencing method described above can be applied in diagnosing or prognosing a disease. For example, the pattern of somatic mutations can be used to predict and/or determine the tissue of origin for a cancer. In some aspects, the genome-wide density of somatic mutations identified from a DNA sample is used for diagnosing a cancer. Other disease that can be diagnosed and/or prognosed using this method will be appreciated in view of this disclosure.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

This Example describes a method for targeted sequencing, and therefore identification, of somatic mutations present in an individual. This method relies on the property of an agent that recognizes and binds specifically to a mismatch in double-stranded DNA. As presented in the following paragraphs, the method can use a mismatch binding protein, MutS (Su and Modrich 1986; Modrich 1994), as the mismatch recognizing enzyme. As the method described in this Example to induce mismatches generates mismatches at both sites of germline variants (inherited from parents) and somatic mutations (arose over the life span of the individual), additional steps for removing germline variants were incorporated. In addition, this Example utilizes massively parallel sequencing technology to position, to identify, and to quantify the occurrence frequency of somatic mutations. The Example here describe a method of identifying genomic mutations that can utilize the Illumina TruSeq platform for massively parallel sequencing. The method in this Example is applicable to any source of genomic DNA, including, but not limited to, samples collected from a human patient or from an animal. The DNA sample can be obtained from any cell source or body fluid, including, but not limited to, blood, urine, saliva, tissue sample collected from biopsy, cerebrospinal fluid, plasma, and serum.

In this Example the method can include the following steps:

1. Genomic DNA fragmentation

2. Heteroduplex formation

3. Mismatch recognition

4. Removing homoduplex (unprotected)

5. Adaptor ligation and library amplification

6. Massively parallel sequencing and mapping

Genomic DNA fragmentation. Genomic DNA of an individual can be first prepared from a cell source (or body fluid) using a standard method well known in the art. The genomic DNA can then be fragmented to a suitable size for duplex formation and for massively parallel sequencing. The fragmentation step can be achieved by using various methods well known in the art, including, but not limited to, restriction enzyme digestion, sonication, and heat fragmentation. Mse I can be used to fragment the genomic DNA to a pool of double stranded DNA fragments of a median size of about 200 bps. The Mse I digestion can be performed in solution. The reaction can be performed in NEB CutSmart Buffer (50 mM Potassium Acetate, 20 mM Tris-acetate, 10 mM Magnesium Acetate, and 100 μg/ml BSA at pH 7.9) for an hour at about 37° C. in a total reaction volume of 50 μl. Each unit of Mse I can be used to digest 1 μg of genomic DNA. Mse I digestion can also create for each DNA fragment an AT overhang at its 5′ end. The overhang can created by design for the downstream step of sequencing adaptor ligation. Alternative restriction enzymes could be used for this fragmentation step provided that appropriate modifications are made to the design of the sequencing adaptor.

Heteroduplex formation. Following fragmentation of the genomic DNA, double stranded DNA fragments can be first heat denatured and then re-annealed to form either hetero- or homo-duplex. The denaturation and re-annealing step can create mismatches in heteroduplex DNA. These heteroduplex DNA molecules are formed between two strands of DNA molecules that are different either at the site of a somatic mutation or at the site of a germline variant (i.e. a site where the paternal copy of the genome differ from the maternal copy). FIG. 3 illustrates the types of duplexes that could arise from re-annealing DNA fragments prepared from a single individual. Since somatic mutations are rare, after heat denaturation, DNA molecules harboring a somatic mutation is much more likely to anneal with a DNA molecule (of almost complementary sequence) without the same mutation and form a heteroduplex than to anneal with a DNA molecule of exact complementary sequence. The probability of forming a heteroduplex is determined largely by the allele frequency of the mutation. For example, for DNA molecules harboring germline variants (allele frequency of 0.5), there is a 50% chance of heteroduplex formation each time the DNA molecule is denatured and re-annealed.

A heteroduplex can be formed by any method of hybridization known in the art. The denaturing and re-annealing condition can be follows. Mse I digested genomic DNA fragments are first “cleaned up” using a silica column-based kit or phenol-chloroform extraction or any one of the “clean up” methods well known in the art. The “cleaned” DNA is resuspended in 45 μl of water. To create a buffer condition appropriate for the proposed denaturation and re-annealing temperature shifts, 5 μl of 10× PCR buffer [100 mM Tris-HCl (pH 8.8 at 25° C.), 500 mM KCl, 16 mM MgCl2, and 1.0% Triton X-100] is added to the resuspended DNA solution. For the actual denaturing and re-annealing of double stranded DNA fragments. The DNA solution can be first heated up to 98° C. and incubate for 5 min. The reaction can be then allowed to cool down gradually to 25° C. in six successive steps (Velasco et al. 2007): first cooling to 90° C. over 5 min, followed by cooling to 80° C. over 5 min, followed by cooling to 75° C. over 10 min, followed by cooling to 60° C. over 10 min, followed by cooling to 40° C. over 20 min, and finally cooling to 25° C. over 20 min. If working with PCR amplified fragments of DNA, the same “clean up” procedure and temperature-shifting conditions can be applied to denature and to re-anneal the PCR products. The stringency of the hybridization conditions can be optimized as desired, by e.g. adjusting temperature and/or salt concentration of the buffer.

Mismatch recognition. In this step, the duplex (homo- or hetero-) DNA molecules can be treated with an agent having the ability to bind specifically to mismatched base pairs. This includes, but is not limited to, mismatch binding proteins. The agent is contacted to DNA under conditions that allow binding of the agent specifically to mismatch regions. Histidine-tagged recombinant Thermus Thermophilus MutS proteins (Stanislawska-Sachadyn et al. 2003) can be incubated with duplex DNA fragments in solution (resuspended in the buffer condition described in the previous section) at 60° C. for 5 min to form DNA-MutS complex. The precise quantity of MutS protein to apply can be empirically determined using techniques known to one of ordinary skill in the art. Typically about 10-fold molar excess of the number of DNA fragments can be used. Similarly, the incubation temperature and duration could be optimized to achieve the desired base pairing stringency for target DNA. The pH of incubation condition affects sensitivity and specificity of the assay. Bias towards detection of G:T mismatches has previously been reported at higher pH (Stefano 2001). Thus the pH of incubation condition can be optimized to achieve the desired strength and bias of binding. In addition, supplements of magnesium ions or ATP can be included in the reaction to further optimize the binding condition (Stefano 2001).

Unless the DNA sample is prepared from an inbred individual, at this step, the DNA heteroduplexes would mostly contain germline variants. Somatic mutations will make-up only a minor proportion of the heteroduplexes recovered. To focus downstream sequencing efforts on identifying somatic mutations, much of the parental heteroduplexes can be removed by hybridization to DNA probes targeting regions of the genome that contain common SNPs (1000 Genomes Project Consortium et al. 2015). These probes can be synthesized by oligonucleotide synthesizer or be prepared from an appropriate cell line. An appropriate cell line is heterozygous at most of the common SNPs that are relevant to the test subject.

To generate DNA probes from a cell line, genomic DNA can be prepared from the cell line and can be subjected to fragmentation, heteroduplex formation, and mismatch recognition processes similar to the above described. MutS bound heteroduplex DNA fragments are then eluted and biotinylated. Biotinylated DNA fragments are used as hybridization probes to remove heteroduplexes that are homologous in sequences to the probe. The probes can be mixed with fragmented genomic DNA prepared from the test subject (after the “clean up” step) in 10-fold molar excess. The mixture ca then go through the denature-reannealing process to hybridize between the probes and genomic DNA fragments that contain the defined set of common SNPs. The probe-genomic DNA hybridization can occur simultaneously with the induction of heteroduplex formation, which is described in step 2: “Heteroduplex formation” (Box 3 in FIG. 2). These probe-genomic DNA hybrids can then be removed from the pool of heteroduplexes by affinity purification. While genetic variations between the test subject and the cell line chosen for probe generation will lead to residual parental heteroduplexes, careful selection of cell lines or a mixture of cell lines could essentially cover all parental variations. Alternatively, cell lines derived from the test subject can be used as the source for probe generations. However, as will be discussed in the “Massively parallel sequencing and mapping” section below, when estimating allele frequencies of somatic mutations, some residual parental heteroduplexes can be included.

Removing homoduplex. This step is can enrich the pool of genomic DNA fragments with fragments that contain mutations in order to focus the downstream sequencing and mapping efforts. This can be achieved by removing homoduplexes from the pool of DNA fragments before sequencing. After contacting duplex DNA with an agent having the ability to bind specifically to mismatched base pairs in the previous step, heteroduplex DNA molecules are distinct from homoduplex by labeling or by binding by the agent. In the preferred embodiment of the invention, mismatches on heteroduplexes are bound by complexes of mutS proteins. Homoduplexes can therefore be removed by either exonuclease digestion or by affinity-precipitation of mutS-heteroduplex complex or by other means that would separate protein-DNA complexes from free DNA. Heteroduplexes can be first precipitated from the solution by affinity binding, and then treated with exonuclease to further clean up the precipitated heteroduplexes (FIG. 2). For the affinity-binding step, histidine-tagged mutS-heteroduplex complexes are first precipitated from the solution by binding to nickel-conjugated magnetic beads (His-Pur, Thermo Scientific). Free homoduplex DNA fragments are washed away and then mutS-heteroduplex complexes could be eluted by applying a saturated amount of imidazole to compete for binding to nickel-conjugated magnetic beads. The precipitated heteroduplexes can be further treated with exonuclease to remove residual homoduplexes and to remove exposed (unprotected) regions of the heteroduplex (e.g. regions not covered by MutS complexes). The exonuclease treatment is done before elution of the mutS-DNA complex from the nickel-conjugated magnetic beads. The precipitated heteroduplexes can be treated with T5 exonuclease at 37° C. for about 30 min on a nutator. T5 exonuclease digests both single stranded and double stranded DNA substrates from the 5′ to the 3′ end. This polarity of digestion was chosen to preserve the regions 3′ to the mutS complex on each DNA fragment (FIG. 5). Since mutS complexes covers only about 30 nucleotide (nt) region (Obmolova et al. 2000; Lamers et al. 2000) of the heteroduplex DNA, treating mutS-DNA complex with nucleases that don't have polarity of digestion can result in short DNA fragments for sequencing. With a mutation in the middle of the fragment, this can impose difficulties to mapping the short reads to the genome. The region preserved by T5 exonuclease (i.e. regions 3′ to the mutS complex) of each fragment, on the other hand, is likely to more than double the read length and will therefore increase the mappability of sequencing reads. However, T5 exonuclease digestion imposes difficulties for ligation of sequencing adaptors: single stranded DNA ends are not ideal substrates for ligase and ligation to the recessed ends requires either prior knowledge on the sequence of each recessed end or complicated adaptor designs. Thus, adaptor ligation to duplex DNA fragments can be performed at the step immediately before T5 exonuclease digestions (See FIG. 2 for a flow chart to clarify the order of individual steps). Details on the design of sequencing adaptors and ligation procedures are described in the following section.

Adaptor Ligation and Library Amplification.

In order to adapt a high-throughput sequencing approach for identifying mutations captured by mutS (or an alternative mutation recognition agent used), sequencing adaptors can be attached to the mutS-bound heteroduplexes. These sequencing adaptors provide universal priming sites for amplification and sequencing of all heteroduplexes isolated. This Example adapt the Illumina TruSeq platform. The adaptor can be designed with the P5 and P7 priming sites for bridge amplification (Boles and Abrams 2002) and SP1 and SP2 for sequencing (FIG. 7). In addition, a library index for multiplexing and a unique molecular identifier (UMI) can be incorporated for minimizing experimental artifacts in quantification. Finally, all these features can be designed on a single strand of DNA while the complementary strand of the adaptor is a short DNA segment placed to facilitate double-stranded DNA ligation (FIG. 7). The adaptor can be ligated to heteroduplex using T4 DNA ligase. As described in the previous section, this is done after the mutS-heteroduplex precipitation step and before T5 exonuclease digestion. To avoid ligation between heteroduplex DNA fragments, mutS-heteroduplex complexes are treated with Calf Intestinal Alkaline Phosphatase to remove the 5′ phosphate from DNA. The phosphatase reaction can be performed in NEB CutSmart Buffer (50 mM Potassium Acetate, 20 mM Tris-acetate, 10 mM Magnesium Acetate, and 100 μg/ml BSA at pH 7.9) at 37° C. for 15 min. Adaptors are then incubated with phosphatase-treated mutS-heteroduplex complexes and T4 DNA ligase in T4 DNA ligase reaction buffer (50 mM Tris-HCl, 10 mM MgCl₂, 1 mM ATP, 10 mM DTT, pH 7.5) at about 16° C. for 30 min. The adaptor strand can be ligated to the 3′ end of each strand of the heteroduplex (FIG. 5) and the subsequent T5 exonuclease digestion can remove the complementary strand (FIG. e). After exonuclease digestion, DNA is isolated from the mutS protein complex and then circularized to bring the SP1 priming site close to the site of the mutation (FIG. 5). This step can be achieved by incubating the single stranded DNA elutes with CircLigase (epicentre, catalog No. CL4111K) in CircLigase reaction buffer, which is supplemented with ATP and MnCl₂, at 60° C. for an hour. Finally, the circularized DNA molecules are used as templates in a PCR with P5 and P7 primers. This step amplifies the single-stranded circular DNA templates to produce double-stranded linear sequencing libraries for Illumina TruSeq platform. The exact condition of the thermal cycling should be optimized by a skilledipractitioner of the art. This optimization process will benefit from following Illumina's recommendations on P5 and P7 primers and observing the expected average insert size of the sequencing library.

Massively parallel sequencing and mapping. In order to identify mutations captured by mutS protein complexes, the final step of this method is to determine the base sequence of each strand of each heteroduplex DNA fragment. The Illumina TruSeq platform for next generation sequencing can be used. After adaptor ligation and PCR amplification, as described in the previous section, the sequencing libraries can be loaded onto a flow cell and bridge amplified according to the Illumina protocol. Bridge amplification creates clusters of DNA fragments of identical sequences (Boles and Abrams 2002), which allows the sequence by synthesis approach (Bentley et al. 2008) to sequentially image incorporation of individual nucleotides. These sequential images can then be processed and converted to sequence files using software pipelines made and maintained by Illumina. These sequence files can be in the FASTQ format (Cock et al. 2010). Each sequencing read can then be mapped to the appropriate genome build using an alignment program. When processing human samples, sequencing reads can be mapped to the latest release of the human genome build using programs such as Bowtie (Langmead and Salzberg 2012) or BWA (Li and Durbin 2009).

After aligning sequencing reads to the genome, occurrence of each mutation in the sample can be estimated by counting the number of sequencing reads that carry the exact mutation. This can then be converted to a relative allele frequency by comparing the number of sequencing reads carrying each somatic mutation to the number of sequencing reads carrying germline variants. Some normalization or standardization is likely required to make unbiased estimates of allele frequency. MutS proteins, and other mutation recognition agents, are known to have biases in binding affinities to different types of mismatches (Su and Modrich 1986). In addition, these biases are sensitive to the exact experimental condition during the binding/recognition step (Stefano 2001). Moreover, uneven sequencing coverage across the genome will further complicate the count-based estimate of allele frequency.

In order to attain unbiased estimates of allele frequency for somatic mutations, appropriate assumptions and normalization schemes can be utilized. These assumptions and normalization schemes can make use of the shared allele frequency among germline variants (50%) and the potential sharing of sequencing coverage between proximal genomic regions. Application in disease diagnosis and prognosis. The allele frequencies of somatic mutations, the genome wide distributions of somatic mutations, and other patterns of somatic mutations identified by the sequencing method described above can be applied to provide diagnosis and prognosis for diseases. This Example focuses on analysis of plasma DNA samples prepared from cancer patients. Although additional patterns derived from sequencing somatic mutations can be useful for diagnosis and prognosis of cancer, this Example focuses on using allele frequency and genome wide distributions of somatic mutations. Distribution of somatic mutations across the genome, in other words, density of somatic mutations along the genome, has been shown to correlate with features in chromatin modifications (Polak et al. 2015; Schuster-Böckler and Lehner 2012).

For example, Polak et al demonstrated that in cancer cells, local density of somatic mutations is correlated with accessibility of local chromatin (Polak et al. 2015). Since genome-wide chromatin accessibility patterns are distinct between different cell types, using a machine learning approach, they further demonstrated that, it is possible to predict the tissue of origin for a cancer sample based on the patterns of somatic mutations (Polak et al. 2015). The genome-wide density of somatic mutations that can be identified from plasma DNA sample can be used for cancer diagnosis.

In this Example, where the genome-wide density of somatic mutations, which are identified from plasma DNA sample, highly resembles the genome-wide chromatin accessibility pattern of a liver cell. This observation indicates the presence of DNA from dead liver (possibly cancerous) cells in the blood stream. In this scenario, the method of this Example can allow for a liver cancer diagnosis for the patient with corresponding probabilities. The probability associated with this diagnosis is largely based on model fit, while the confidence in the probability reported will depend largely on the nature of the signal and on data quality. Other information on the patient can also be considered. For example, if the patient is known to have liver disease, the observed somatic mutation pattern in plasma DNA could then instead reflect DNA released from dead liver cells resulted from the said liver disease. In this case, a more careful evaluation will be required before providing a diagnosis. The exact implementation of the application and the exact form of the model based on this Example can be determined empirically by a skilled practitioner of the art.

In some instances, the relative allele frequencies for each somatic mutation can be leveraged to provide a prognosis for cancer progression. For example, following the liver cancer scenario, a patient with a higher allele frequency of a group of mutations, that have a density pattern resembles chromatin accessibility of a liver cell, would indicate a more progressed liver cancer than a patient with a lower allele frequency of somatic mutations of the same, or similar, density pattern.

### REFERENCES FOR EXAMPLE 1

1000 Genomes Project Consortium, Auton A, Brooks L D, Durbin R M, Garrison E P, Kang H M, Korbel J O, Marchini J L, McCarthy S, McVean G A, et al. 2015. A global reference for human genetic variation. Nature 526: 68-74.

Alioto T S, Buchhalter I, Derdak S, Hutter B, Eldridge M D, Hovig E, Heisler L E, Beck T A, Simpson J T, Tonon L, et al. 2015. A comprehensive assessment of somatic mutation detection in cancer using whole-genome sequencing. Nature Communications 6: 10001.

Bentley D R, Balasubramanian S, Swerdlow H P, Smith G P, Milton J, Brown C G, Hall K P, Evers D J, Barnes C L, Bignell H R, et al. 2008. Accurate whole human genome sequencing using reversible terminator chemistry. Nature 456: 53-59.

Bettegowda C, Sausen M, Leary R J, Kinde I, Wang Y, Agrawal N, Bartlett B R, Wang H, Luber B, Alani R M, et al. 2014. Detection of Circulating Tumor DNA in Early- and Late-Stage Human Malignancies. Sci Transl Med 6: 224ra24.

Boles T, Abrams E. 2002. Solid phase methods for amplifying multiple nucleic acids. US Pat. Pub. No.: 2002/0132245.

Canard B, Sarfati S. 1994. Novel derivatives for use in nucleic acid sequencing. International Pat. Pub. WO 1994023064 A1.

Cibulskis K, Lawrence M S, Carter S L, Sivachenko A, Jaffe D, Sougnez C, Gabriel S, Meyerson M, Lander E S, Getz G. 2013. Sensitive detection of somatic point mutations in impure and heterogeneous cancer samples. Nat Biotech 31: 213-219.

Cock P J A, Fields C J, Goto N, Heuer M L, Rice P M. 2010. The Sanger FASTQ file format for sequences with quality scores, and the Solexa/Illumina FASTQ variants. Nucleic Acids Res 38: 1767-1771.

Goodwin S, McPherson J D, McCombie W R. 2016. Coming of age: ten years of next-generation sequencing technologies. Nat Rev Genet 17: 333-351.

Kivioja T, Vähärautio A, Karlsson K, Bonke M, Enge M, Linnarsson S, Taipale J. 2012. Counting absolute numbers of molecules using unique molecular identifiers. Nat Meth 9: 72-74.

Lamers M H, Perrakis A, Enzlin J H, Winterwerp H H, de Wind N, Sixma T K. 2000. The crystal structure of DNA mismatch repair protein MutS binding to a G×T mismatch. Nature 407: 711-717.

Langmead B, Salzberg S L. 2012. Fast gapped-read alignment with Bowtie 2. Nat Meth 9: 357-359.

Li H, Durbin R. 2009. Fast and accurate short read alignment with Burrows-Wheeler transform. Bioinformatics 25: 1754-1760.

Lohr J G, Adalsteinsson V A, Cibulskis K, Choudhury A D, Rosenberg M, Cruz-Gordillo P, Francis J M, Zhang C-Z, Shalek A K, Satija R, et al. 2014. Whole-exome sequencing of circulating tumor cells provides a window into metastatic prostate cancer. Nat Biotech 32: 479-484.

Mardis E R. 2013. Next-Generation Sequencing Platforms. Annu Rev anal Chem. 6:287-303.

Modrich P. 1994. Mismatch repair, genetic stability, and cancer. Science 266: 1959-1960.

Newman A M, Bratman S V, To J, Wynne J F, Eclov N C W, Modlin L A, Liu C L, Neal J W, Wakelee H A, Merritt R E, et al. 2014. An ultrasensitive method for quantitating circulating tumor DNA with broad patient coverage. Nat Med 20: 548-554.

Obmolova G, Ban C, Hsieh P, Yang W. 2000. Crystal structures of mismatch repair protein MutS and its complex with a substrate DNA. Nature 407: 703-710.

Polak P, Karlić R, Koren A, Thurman R, Sandstrom R, Lawrence M S, Reynolds A, Rynes E, Vlahovic̆ek K, Stamatoyannopoulos J A, et al. 2015. Cell-of-origin chromatin organization shapes the mutational landscape of cancer. Nature 518: 360-364.

Schuster-Böckler B, Lehner B. 2012. Chromatin organization is a major influence on regional mutation rates in human cancer cells. Nature 488: 504-507.

Stanislawska-Sachadyn A, Sachadyn P, Jedrzejczak R, Kur J. 2003. Construction and purification of his6-Thermus thermophilus MutS protein. Protein Expr Purif 28: 69-77.

Stefano J E. 2001. Method for detecting and identifying mutations. U.S. Pat. No. 6,297,010.

Su S S, Modrich P. 1986. Escherichia coli mutS-encoded protein binds to mismatched DNA base pairs. Proc Natl Acad Sci USA 83: 5057-5061.

Velasco E, Infante M, Durán M, Pérez-Cabornero L, Sanz D J, Esteban-Carden̆osa E, Miner C. 2007. Heteroduplex analysis by capillary array electrophoresis for rapid mutation detection in large multiexon genes. Nat Protoc 2: 237-246.

Woo Y H, Li W-H. 2012. DNA replication timing and selection shape the landscape of nucleotide variation in cancer genomes. Nature Communications 3: 1004.

We claim:

1. A method of identifying genomic DNA mutations, the method comprising:
denaturing double stranded (ds) genomic DNA fragments to form single stranded (ss) genomic DNA fragments;
annealing the ssDNA to form homoduplex DNA fragments and heteroduplex DNA fragments;
enzymatically identifying a heteroduplex DNA fragment having a base pair mismatch by binding the heteroduplex DNA fragment having a base pair mismatch;
separating the heteroduplex DNA fragment having a base pair mismatch from the homoduplex DNA fragments and any heteroduplex DNA not having a base pair mismatch to obtain an isolated heteroduplex DNA fragment having a base pair mismatch;
ligating a sequencing adaptor to an end of each strand of the isolated heteroduplex DNA fragment having a base pair mismatch;
digesting the adaptor-ligated isolated heteroduplex DNA fragment having a base pair mismatch with an exonuclease capable of degrading ds DNA
removing the enzyme from the adaptor-ligated heteroduplex DNA fragment having a base pair mismatch and forming an adaptor-ligated ss DNA;
circligating the adaptor-ligated ss DNA;
PCR amplifying the circle-ligated adaptor-ligated ssDNA to form a sequencing library; and
sequencing the sequencing library using a next generation sequencing method.

2. The method of claim 1, wherein the step of enzymatically identifying a heteroduplex DNA fragment having a base pair mismatch comprises binding the heteroduplex DNA fragment having a base pair mismatch with an enzyme that specifically binds the heteroduplex DNA fragment at mismatched base pair.

3. The method of claim 2, wherein the enzyme is mutS.

4. The method of claim 3, wherein the mutS comprises an affinity purification tag.

5. The method of claim 4, wherein the affinity purification tag is selected from the group consisting of: a his-tag, a chitin binding protein tag, maltose biding protein tag, a strep-avidin tag, a glutathione-S-transferase tag, FLAG-tag, V5-tag, VSV tag Myc-tag, HA0tag, Spot tag, NE tag and any combination thereof.

6. The method of claim 5, wherein the step of separating is performed by affinity precipitating out the heteroduplex DNA with an antibody or affinity purification complex that specifically binds the mutS or affinity purification tag.

7. The method of claim 6, wherein the exonuclease is T5 exonuclease.

8. The method of claim 1, wherein the next generation sequencing method is a sequencing by ligation method or a sequencing by synthesis method.

9. The method of claim 8, wherein the sequencing by synthesis method is pyrosequencing.

10. The method of claim 1, wherein the exonuclease is T5 exonuclease.

11. The method of claim 1, wherein ds genomic fragments are from genomic DNA of a single subject.

12. The method of claim 11, further comprising digesting the genomic DNA with a restriction enzyme to generate the ds genomic fragments.

13. The method of claim 1, wherein the step of sequencing forms sequencing reads.

14. The method of claim 13, further comprising mapping the sequencing reads to a genome build.

15. The method of claim 14, further comprising the step of identifying mismatch variants and estimating relative allele frequency (RAF) and identifying somatic mutations from parental mutations.

16. The method of claim 1, wherein the genomic DNA mutation is a somatic mutation.

17. A method of identifying genomic DNA mutations, the method comprising:
denaturing double stranded (ds) genomic DNA fragments to form single stranded (ss) genomic DNA fragments;
annealing the ssDNA to form homoduplex DNA fragments and heteroduplex DNA fragments;
identifying a heteroduplex DNA fragment having a base pair mismatch to form an identified heteroduplex DNA fragment;
separating the heteroduplex DNA fragment from the unprotected homoduplex DNA fragments;
ligating an adaptor to an end of each DNA strand in the heteroduplex DNA fragment to form an adaptor-ligated DNA;
PCR amplifying the adaptor-ligated DNA to form a sequencing library; and
sequencing the sequencing library using a next generation sequencing method.

18. The method of claim 17, wherein the step of sequencing forms sequencing reads and further comprises mapping the sequencing reads to a genome build.

19. The method of claim 18, further comprising the step of identifying mismatch variants and estimating relative allele frequency (RAF) and identifying somatic mutations from parental mutations.

20. The method of claim 17, wherein the next generation sequencing method is a sequencing by ligation method or a sequencing by synthesis method.
